(19) United States
(12) Patent Application Publication
(10) Pub. No.: US 2022/0151983 A1
(43) Pub. Date: May 19, 2022

(54) COMPOUNDS FOR INHIBITING FUCOSYLATION AND METHODS FOR USING THE SAME

(71) Applicant: SCORE PHARMA, INC., Exton, PA (US)

(72) Inventor: Bruce E. JONES, Exton, PA (US)

(21) Appl. No.: 17/440,447

(22) PCT Filed: Mar. 16, 2020
(86) PCT No.: PCT/US2020/022896

§ 371 Date: Sep. 17, 2021

Related U.S. Application Data
(60) Provisional application No. 62/819,724, filed on Mar. 18, 2019.

(51) Int. Cl. A61K 31/353; A61K 45/06

(52) U.S. Cl. CPC A61K 31/353; A61K 45/06

(57) ABSTRACT

The disclosure generally provides for compounds for inhibiting fucosylation and methods for using the same and methods of producing antibodies with reduced fucosylation.

### FIELD

The disclosure generally provides compounds for inhibiting the fucosylation of proteins, such as antibodies, and uses thereof.

### BACKGROUND

L-fucose, also referred to as 6-deoxy-L-galactose or just fucose, is a monosaccharide that is a component of some N- and O-linked glycans and glycolipids in animals. Fucose is typically added as a terminal modification to glycans, including glycans attached to blood group antigens, selectins and antibodies. Fucosylation of proteins is believed to play a role in mammalian development, and aberrant protein fucosylation has been proposed to be associated with human disease, including up-regulation in cancers and rheumatoid arthritis. Removal of fucosylation has been shown to improve antibody binding and activity. Thus, there is a need for compounds that can be used to inhibit fucosylation. The present embodiments fulfills this need as well as others.

### SUMMARY

In some embodiments, methods of inhibiting fucosyltransferase are provided. In some embodiments, the methods comprise contacting a fucosyltransferase with a composition comprising purpurogallin, hexachlorophene, acriflavinum, baicalein, epicatechin monogallate, epicatechin-3-monogallate, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof. In some embodiments, the fucosyltransferase is in a cell that produces an antibody.

In some embodiments, methods of inhibiting fucosylation of a protein in a cell are provided. In some embodiments, the methods comprise contacting the cell with a composition comprising purpurogallin, hexachlorophene, acriflavinum, baicalein, epicatechin monogallate, epicatechin-3-monogallate, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof, to inhibit the fucosylation of the protein. In some embodiments, the composition inhibits fucosylation by inhibiting a fucosyltransferase.

In some embodiments, methods of producing an antibody with reduced fucosylation are provided. In some embodiments, the methods comprise contacting a cell producing an antibody with a composition comprising purpurogallin, hexachlorophene, acriflavinum, baicalein, epicatechin monogallate, epicatechin-3-monogallate, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof. In some embodiments, the cell is contacted with the composition in vitro or in vivo. In some embodiments, the antibody is secreted from the cell. In some embodiments, the compound contacts a fucosyltransferase prior to the secretion of the antibody.

In some embodiments, methods of producing an antibody with an altered glycosylation pattern are provided. In some embodiments, the methods comprise contacting a cell producing the antibody with a composition comprising purpurogallin, hexachlorophene, acriflavinum, baicalein, epicatechin monogallate, epicatechin-3-monogallate, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof. In some embodiments, the cell is contacted with the composition in vitro or in vivo. In some embodiments, the antibody is secreted from the cell. In some embodiments, the compound contacts a fucosyltransferase prior to the secretion of the antibody. An “altered glycosylation pattern” in reference to a protein, such as an antibody produced according to any of the methods described herein, refers to the protein having a different glycosylation pattern compared to an protein produced from the same cell that was not contacted with the composition or compound described herein.

In some embodiments, compositions comprising purpurogallin, hexachlorophene, acriflavinum, baicalein, epicatechin monogallate, epicatechin-3-monogallate, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof, and a cell are provided.

In some embodiments, compositions comprising purpurogallin, hexachlorophene, acriflavinum, baicalein, epicatechin monogallate, epicatechin-3-monogallate, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof, and an antibody are provided.

In some embodiments, cell free media comprising purpurogallin, hexachlorophene, acriflavinum, baicalein, epicatechin monogallate, epicatechin-3-monogallate, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof are provided. In some embodiments, the media further comprises an antibody.

### BRIEF DESCRIPTION OF THE DRAWINGS

FIG. 1 depicts a graph of inhibition fucosylation using purpurogallin.

FIG. 2 depicts a graph of inhibition fucosylation using hexachlorophene.

FIG. 3 depicts a graph of inhibition fucosylation using acriflavinium.

FIG. 4 depicts a graph of inhibition fucosylation using epicatechin monogallate.

FIG. 5 depicts a graph of inhibition fucosylation using epicatechin-3-monogallate.

FIG. 6 depicts a graph of inhibition fucosylation using epicatechin-3,5-digallate.

FIG. 7 depicts a graph of inhibition fucosylation using theaflavin monogallate.

FIG. 8 depicts a graph of inhibition fucosylation using tannic acid.

FIG. 9 depicts a graph of inhibition fucosylation using methacycline.

FIG. 9 depicts a graph of inhibition fucosylation using methacycline.

FIG. 9 depicts a graph of inhibition fucosylation using methacycline.

FIG. 10 depicts a graph of inhibition fucosylation using mitoxanthrone hydrochloride.

FIG. 11 depicts a graph of inhibition fucosylation using hycanthone.

FIG. 12 depicts a graph of inhibition fucosylation using etharidine lactate.

FIG. 13 depicts a graph of inhibition fucosylation using aurin.

FIG. 14 depicts a graph of inhibition fucosylation using carboplatin.

FIG. 15 depicts a graph of inhibition fucosylation using cisplatin.

FIG. 16 depicts a graph of inhibition fucosylation using diometin.

FIG. 17 depicts a graph of inhibition fucosylation using suramin.

FIG. 18 depicts a graph of inhibition fucosylation using hematein.

FIG. 19 depicts a graph of inhibition fucosylation using gossypol.

FIG. 20 depicts a reduction of antibody fucosylation by Epicatechin Monogallate (ECG).

FIG. 21 depicts a reduction of antibody fucosylation by Epicatechin-3-Monogallate (ECGC).

### DETAILED DESCRIPTION

As used herein and unless otherwise indicated, the term “about” is intended to mean±5% of the value it modifies. Thus, about 100 means 95 to 105.

As used herein and in the appended claims, the singular forms “a”, “an” and “the” include plural reference unless the context clearly dictates otherwise.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

The term “antibody” includes immunoglobulin or antibody molecules including polyclonal antibodies, monoclonal antibodies including murine, human, humanized and chimeric monoclonal antibodies and antibody fragments. In some embodiments, the antibody is a recombinant antibody. A “recombinant antibody” refers to antibody that is expressed from a cell that has been genetically modified to produce a specific antibody. Methods of producing recombinant antibodies are known and any such method can be used.

In some embodiments, an antibody refers to a polypeptide that exhibit binding specificity to a specific antigen or target molecule. Intact antibodies are heterotetrameric glycoproteins, composed of two light chains and two heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa and lambda, based on the amino acid sequences of their constant domains. In some embodiments, the antibody is IgA, IgD, IgE, IgG and IgM type antibody. In some embodiments, the antibody is a IgA₁, IgA₂, IgG₁, IgG₂, IgG₃ and IgG₄ type antibody.

In some embodiments, the antibody is an antibody fragment. The term “antibody fragment” means a portion of an intact antibody, generally the antigen binding or variable region of the intact antibody. Examples of antibody fragments include, but are not limited to, Fab, Fab′, F(ab′)₂ and Fv fragments, diabodies, single chain antibody molecules and multispecific antibodies that bind to multiple targets or to different epitopes of the same target.

In some embodiments, the antibody is an antibody-drug conjugate. The term “antibody-drug conjugate” means an antibody or an antibody fragment attached to a drug. Examples of antibody-drug conjugates include, but are not limited to, brentuximab vedotin, cantuzumab rautansine, gemtuzumab ozogamicin, ibritumomab tiuxetan, poltuzumab vedotin, sacituzumab govitecan, trastuzumab duocarmazine, trastuzumab emtansine, and inotuzumab ozogamicin.

Polyclonal antibodies are heterogeneous populations of antibody molecules derived from the sera of animals immunized with an antigen. See, for example Kohler and Milstein, Nature 256:495 497 (1975); U.S. Pat. No. 4,376,110; Ausubel et al., eds., Current Protocols in Molecular Biology, Greene Publishing Assoc. and Wiley Interscience, N.Y., (1987, 1992); and Harlow and Lane ANTIBODIES: A Laboratory Manual Cold Spring Harbor Laboratory (1988); Colligan et al., eds., Current Protocols in Immunology, Greene Publishing Assoc. and Wiley Interscience, N.Y., (1992, 1993), the contents of which references are incorporated entirely herein by reference. Such antibodies may be of any immunoglobulin class including IgG, IgM, IgE, IgA, IgD and any subclass thereof. A hybridoma producing a mAb of the present invention may be cultivated in vitro, in situ or in vivo. Production of high titers of mAbs in vivo or in situ makes this the presently preferred method of production.

The term “monoclonal antibody” (mAb) as used herein means an antibody (or antibody fragment) obtained from a population of substantially homogeneous antibodies. Monoclonal antibodies are typically being directed against a single antigenic determinant. The modifier “monoclonal” indicates the substantially homogeneous character of the antibody and does not require production of the antibody by any particular method. For example, murine mAbs can be made by the hybridoma method of Kohler et al., Nature 256:495-497 (1975). Chimeric mAbs containing a light chain and heavy chain variable region derived from a donor antibody (such as, but not limited to, murine) in association with light and heavy chain constant regions derived from an acceptor antibody (such as another mammalian species, including but not limited to human) can be prepared by the method disclosed in U.S. Pat. No. 4,816,567 or any other methods. Humanized mAbs having CDRs derived from a non-human donor immunoglobulin (such as, but not limited to, murine) and the remaining immunoglobulin-derived parts of the molecule being derived from one or more human immunoglobulins, optionally having altered framework support residues to preserve binding affinity, can be obtained by the techniques disclosed in Queen et al., Proc. Natl. Acad. Sci. (USA), 86:10029-10032 (1989) and Hodgson et al., Bio/Technology, 9:421 (1991). Methods of humanizing antibodies are routine and not critical to the methods described herein.

The term “in combination with” as used herein means that the described agents can be administered to, or contacted with, a cell, an animal, or target together in a mixture, concurrently as single agents or sequentially as single agents in any order.

As used herein the term “contacting” refers to bringing two components in proximity with one another. In some embodiments, the components can be mixed together or simply placed in the same container. A composition can also be considered to be contacted with another component (e.g. subject, cell, and the like) if it is being administered to the other component. Administration can be through injection, pipetting, and other routine methods of administration.

As used herein, the term “inhibiting,” in reference to enzymatic activity means reducing by any measurable amount the activity of the enzyme.

As used herein, the term “reducing,” in reference to protein modifications means reducing by any measurable amount the amount of the modification.

In some embodiments, the compounds described herein are salts of the compound. In some embodiments, the salt is a pharmaceutically acceptable salt thereof. As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present invention also includes quaternary ammonium salts of the compounds described herein, where the compounds have one or more tertiary amine moiety.

The term “antigen” refers to the target that the antibody binds to.

The term “purified” with reference to a protein or antibody refers to an a protein or antibody that is substantially free of other material that associates with the molecule in its natural environment or cellular environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80%-90% (w/w) pure, 90-95% pure; and, at least 95%, 96%, 97%, 98%, 99%, or 100% (w/w) pure. In some embodiments, the antibody that is produced according to any of the embodiments described herein is further purified after being produced.

Embodiments provided herein provide compositions and methods that can be used, for example, for reducing protein fucosylation. “Reduced fucosylation” in the context of proteins generally refers to reduced addition of fucose to glycans via α(1,2)-, α(1,3)-, α(1,4) and α(1,6)-linkages. Fucosylation in the context of an antibody refers to addition of fucose (“fucosylation”) to N-acetylglucosamine (“GlcNAc”) at the reducing terminal of an N-linked glycan of an antibody. “Reduced fucosylation” in the context of an antibody refers to a reduction of fucose molecules linked to N-acetylglucosamine (“GlcNAc”) at the reducing terminus of an N-linked glycan of an antibody, as compared to an untreated antibody.

Antibody dependent cellular cytotoxicity (ADCC) is a mechanism of cell-mediated immunity whereby an effector cell of the immune system actively lyses a target cell that has been bound by specific antibodies. ADCC is one of the mechanisms through which antibodies, as part of the immune response, can act to limit and contain infection and disease. It has been shown that monoclonal antibodies that have a reduced amount of fucose in their glycosylation pattern exhibit much higher ADCC activity as compared to normally fucosylated antibodies. Without being bound to any particular theory, the mechanism behind the increased ADCC of a low or no fucose antibody seems to be mediated by an increased affinity of the antibodies modified Fc region to the FcγR of the target cell. Thus, the compounds, compositions, and methods provided herein can provide antibodies with higher ADCC activity.

Certain compounds, or salts thereof, are referenced herein. Table 1 provides the chemical structures of these compounds. Also included are salts of these compounds, such as pharmaceutically acceptable salts of the depicted compounds. In some embodiments, the salt is a HCl salt of the compounds.

TABLE 1

| Compound Name | Structural Formula |
|---|---|
| Purpurogallin | 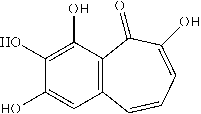 [SMILES: O=c1c(O)cccc2cc(O)c(O)c(O)c12] |
| Hexachlorophene | 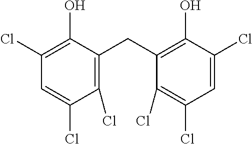 [SMILES: Oc1c(Cl)cc(Cl)c(Cl)c1Cc1c(O)c(Cl)cc(Cl)c1Cl] |
| Acriflavinium | 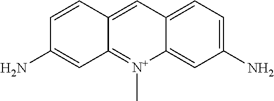 [SMILES: C[n+]1c2cc(N)ccc2cc2ccc(N)cc21] |
| Baicalein | 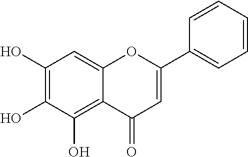 [SMILES: O=c1cc(-c2ccccc2)oc2cc(O)c(O)c(O)c12] |
| Epicatechin Monogallate (ECG) | 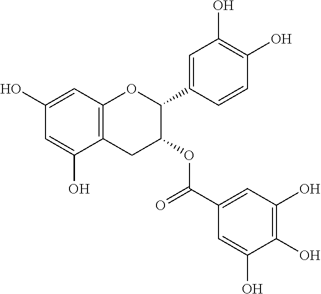 [SMILES: O=C(O[C@@H]1Cc2c(O)cc(O)cc2O[C@@H]1c1ccc(O)c(O)c1)c1cc(O)c(O)c(O)c1] |
| Epicatechin-3- Monogallate (EGCG) | 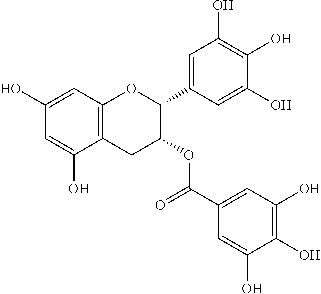 [SMILES: O=C(O[C@@H]1Cc2c(O)cc(O)cc2O[C@@H]1c1cc(O)c(O)c(O)c1)c1cc(O)c(O)c(O)c1] |
| Epicatechin-3,5- Digallate | 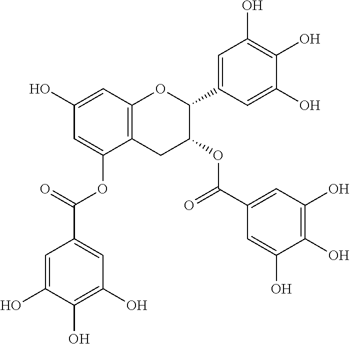 [SMILES: O=C(Oc1cc(O)cc2c1C[C@@H](OC(=O)c1cc(O)c(O)c(O)c1)[C@@H](c1cc(O)c(O)c(O)c1)O2)c1cc(O)c(O)c(O)c1] |
| Theaflavin Monogallate | 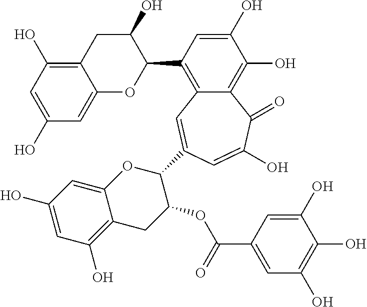 [SMILES: O=C(O[C@@H]1Cc2c(O)cc(O)cc2O[C@@H]1c1cc(O)c(=O)c2c(O)c(O)cc([C@H]3Oc4cc(O)cc(O)c4C[C@H]3O)c2c1)c1cc(O)c(O)c(O)c1] |
| Tannic Acid | 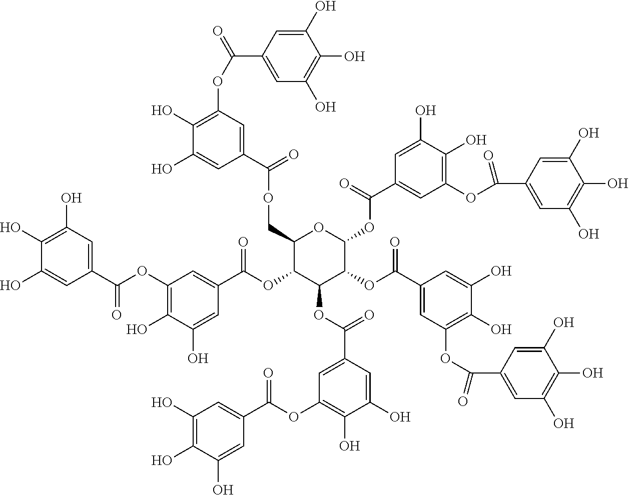 [SMILES: O=C(OC[C@H]1O[C@H](OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1] |
| Methacycline | 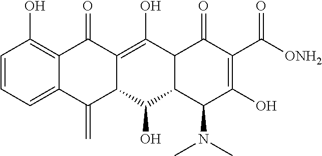 |
| Anthralin | 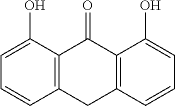 [SMILES: O=C1c2c(O)cccc2Cc2cccc(O)c21] |
| Mitoxanthrone Hydrochloride | 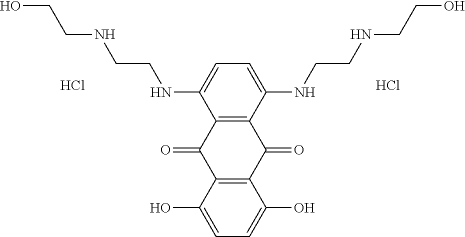 [SMILES: O=C1c2c(O)ccc(O)c2C(=O)c2c(NCCNCCO)ccc(NCCNCCO)c21 ; Cl ; Cl] |
| Hycanthone | 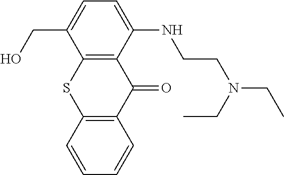 [SMILES: CCN(CC)CCNc1ccc(CO)c2sc3ccccc3c(=O)c12] |
| Etheridine Lactate | 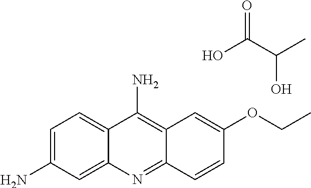 |
| Aurin Tricarboxylic Acid | 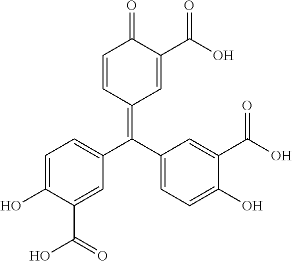 [SMILES: O=C(O)C1=CC(=C(c2ccc(O)c(C(=O)O)c2)c2ccc(O)c(C(=O)O)c2)C=CC1=O] |
| Carboplatin | 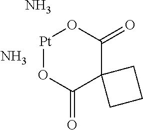 |
| Cisplatin | 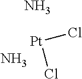 |
| Primuletin | 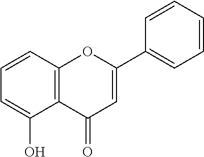 [SMILES: O=c1cc(-c2ccccc2)oc2cccc(O)c12] |
| Chrysin | 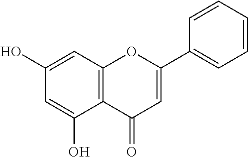 [SMILES: O=c1cc(-c2ccccc2)oc2cc(O)cc(O)c12] |
| Diometin | 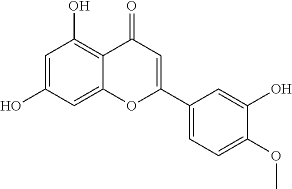 [SMILES: COc1ccc(-c2cc(=O)c3c(O)cc(O)cc3o2)cc1O] |
| Suramin | 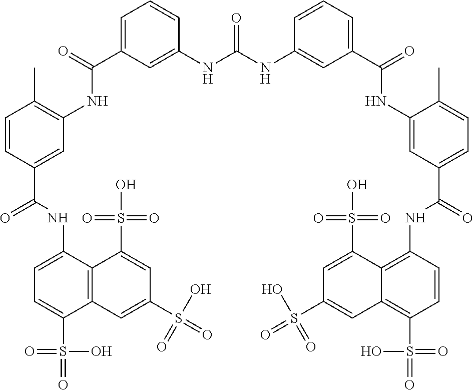 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1] |
| Hematin | 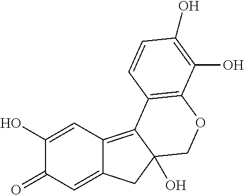 [SMILES: O=C1C=C2CC3(O)COc4c(ccc(O)c4O)C3=C2C=C1O] |
| Gossypol | 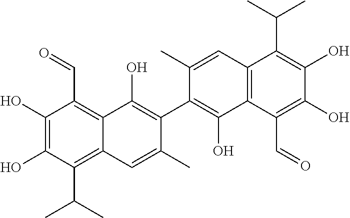 [SMILES: Cc1cc2c(C(C)C)c(O)c(O)c(C=O)c2c(O)c1-c1c(C)cc2c(C(C)C)c(O)c(O)c(C=O)c2c1O] |

In some embodiments, compositions comprising one or more of the compounds are provided herein. In some embodiments, the composition comprises one or more of the compounds and a cell. In some embodiments, the cell is a eukaryotic or prokaryotic cell. In some embodiments, the cell is capable of producing protein. In some embodiments, the cell is a recombinant genetically modified cell. A “recombinant genetically modified cell” is a cell that has been modified with nucleic acid material that is exogenous to the cell, such as by transfection, transduction, or infection. In some embodiments, the cell has been stably transduced or transfected with a nucleic acid molecule encoding an antibody. In some embodiments, the cell produces an antibody.

Various cell or cell types can be used. Non-limiting examples of cells that can be present in the compositions or be used in any of the methods provided for herein a CHO cell, a NSO cell, a Sp2/0 cell, a HEK293 cell, a PER.C6 cell, and the like.

In some embodiments, the compositions further comprise cell culture media. In some embodiments, the compositions comprise selection agents. In some embodiments, the compositions comprise one or more antibiotics. A selection agent is an agent, such as an antibiotic that is used to select for cells that are transfected or transduced with a specific nucleic acid molecule. Various selection agents can be used. Examples, include, but are not limited to, Geneticin™ (G418; (2R,3S,4R,5R,6S)-5-Amino-6-[(1R,2S,3S,4R,6S)-4,6-diamino-3-[(2R,3R,4R,5R)-3,5-dihydroxy-5-methyl-4-methylaminooxan-2-yl]oxy-2-hydroxycyclohexyl]oxy-2-(1-hydroxyethyl)oxane-3,4-diol), hygromycin b (O-6-Amino-6-deoxy-L-glycero-D-galacto-heptopyranosylidene-(1-2-3)-O-β-D-talopyranosyl(1-5)-2-deoxy-N3-methyl-D-streptamine), neomycin, Blasticidin (4-amino-1-[4-({(3S)-3-amino-5-[[amino(imino)methyl](methyl)amino]pentanoyl}amino)-2,3,4-trideoxy-β-D-erythro-hex-2-enopyranuronosyl]pyrimidin-2(1H)-one), Mycophenolic Acid ((E)-6-(4-hydroxy-6-methoxy-7-methyl-3-oxo-1H-2-benzofuran-5-yl)-4-methylhex-4-enoic acid), Zeocin™ (2-({2-[2-{[(6-Amino-2-{3-amino-1-[(2,3-diamino-3-oxopropyl)amino]-3-oxopropyl}-5-methyl-4-pyrimidinyl)carbonyl]amino}-3-[(5-{[1-({2-[4-({4-[(diaminomethylene)amino]butyl}carbamoyl)-4′,5′-dihydro-2,4′-bi-1,3-thiazol-2′-yl]ethyl}amino)-3-hydroxy-1-oxo-2-butanyl]amino}-3-hydroxy-4-methyl-5-oxo-2-pentanyl)amino]-1-(1H-imidazol-5-yl)-3-oxopropoxy}-4,5-dihydroxy-6-(hydroxymethyl)tetrahydro-2H-pyran-3-yl}oxy)-3,5-dihydroxy-6-(hydroxymethyl)tetrahydro-2H-pyran-4-yl carbamate), and puromycin (3′-deoxy-N,N-dimethyl-3′-[(O-methyl-L-tyrosyl)amino]adenosine), or any combination thereof.

In some embodiments, compositions comprising one or more of the compounds provided for herein and an antibody. In some embodiments, the antibody is an antibody secreted and/or produced from a cell. In some embodiments, the antibody is adalimumab, alemtuzumab, alirocumab, atezolizumab, avelumab, belimumab, benralizumab, bevacizumab, bezlotoxumab, blinatumomab, brentuximab vedotin, burosumab, canakinumab, cantuzumab mertansine, cantuzumab ravtansine, carotuximab, certolizumab pegol, cetuximab, claudiximab, daclizumab, daratumumab, denosumab, depatuxizumab mafodotin, dinutuximab, durvalumab, elotuzumab, enfortumab vedotin, enoblituzumab, gemtuzumab ozogamicin, golimumab, guselkumab, ibalizumab, ibritumomab tiuxetan, ifabotuzumab, inebilizumab, infliximab, inotuzumab ozogamicin, ipilimumab, isatuximab, margetuximab, mepolizumab, necitumumab, nimotuzumab, nivolumab, obiloxaximab, obinutuzumab, ocrelizumab, ofatumumab, olaratumab, omalizumab, palivizumab, pembrolizumab, pertuzumab, polatuzumab vedotin, racotumomab, ramucirumab, ranibizumab, raxibacumab, rituximab, rovalpituzumab tesirine, sacituzumab, sacituzumab govitecan, sarilumab, secukinumab, siltuximab, sirukumab, teprotumumab, tildrakizumab, tocilizumab, tositumomab, trastuzumab, trastuzumab duocarmazine, trastuzumab emtansine, ublituxumab, ustekinumab, vedolizumab or a biosimilar thereof.

In some embodiments, the compositions provided herein are cell free or substantially cell free. A composition is substantially cell free if there is less than 50,000, 40,000, 30,000, 20,000, 10,000, or 5,000 cells in the composition. In some embodiments, a composition comprises cell free media comprising one or more of the compounds, or salts thereof, described herein. The cell free media can also comprise any of the other components described herein.

In some embodiments, the antibody is antibody that can bind an ErbB protein, such as but not limited to, ErbB1 also named HER1 or EGFR, ErbB2 also named HER2 or HER2/neu, ErbB3 also named HER3, and ErbB4 also named HER4. In some embodiments, the antibody binds to a Transforming Growth Factor protein, such as but not limited to, TGFB1, TGFB2, TGFB3, and TGFB4. In some embodiments, the antibody binds to a Vascular Endothelial Growth Factor protein, such as but not limited to, VEGFR1, Flt-1, VEGFR2, Flk-1/KDR, VEGFR3, and Flt4. In some embodiments, the antibody binds to Receptor Activator of Nuclear Factor kappa Beta (RANK) also known as TRANCE receptor or TNFRSFIIA. In some embodiments, the antibody binds to proteins related to the Signaling Lymphocyte Activation Molecule Family (SLAMF) of proteins, such as but not limited to, SLAMF1, SLAMF2, SLAMF3, SLAMF4, SLAMF5, SLAMF6, SLAMF7, and SLAMF8. In some embodiments, the antibody binds Platelet Derived Growth Factor Receptor proteins, such as but not limited to, PDGFRα and PDGFRβ. In some embodiments, the antibody binds to the Killer Cell Immunoglobulin Like Receptor (KIR) family of proteins, such as but not limited to, KIR3DL3, KIR3DP1, KIR3DL4, and KIR3DL2. In some embodiments, the antibody binds the Major Histocompatibility Complex Class I Chain Related Protein (MIC), such as but not limited to, MICA and MICB. In some embodiments, the antibody binds to the Tumor Necrosis Factor (TNF) family of proteins, such as but not limited to, LTalpha, LT beta, FASL, 4-IBBL, OX40L, and TNF Related Apoptosis Inducing Ligand (TRAIL). In some embodiments, the antibody binds to the Death Receptor (DR) family of proteins, such as but not limited to, DR1, DR2, DR3, Dr4, DR5, DR6, DR7, and DR8. In some embodiments, the antibody binds to the Cytotoxic T-Lymphocyte Antigen family of proteins, such as but not limited to, CTLA4. In some embodiments, the antibody binds to the Programed Death Receptor, such as but not limited to, PD1. In some embodiments, the antibody binds to the Carcinoembryonic antigen family of proteins, such as but not limited to, CD66a, CD66b, CD66c, CD66d, CD66e, and CD66f. In some embodiments, the antibody binds to the T-Cell Immunoglobulin and mucin-domain family of receptors, such as but not limited to, TIM1, TIM2, TIM3, and TIM4. In some embodiments, the antibody binds to the Lymphocyte activation gene (LAG), such as but not limited to, LAG3. In some embodiments, the antibody binds to the Clusters of Differentiation, such as but not limited to, CD2, CD3, CD19, CD20, CD22, CD25, CD27, CD30, CD 33, CD38, CD39, CD52, CD70, CD73, CD94, CD134, CD137, CD252, and CD340. In some embodiments, the antibody binds to Tumor Associated Carbohydrate Antigens, such as but not limited to, mucin related GalNAc-O-ser/thr also known as Tn and Neu5Acα2-6GalNAcα-O-Ser/Thr also known as Sialyl Tn. In some embodiments, the antibody binds to stage-specific embryonic antigens, such as but not limited to, SSEA-1 (also known as Lewisₓ), SSEA-2, SSEA-3, and SSEA-4. In some embodiments, the antibody binds to the Thomsen-Freidenreich antigens, also known as Gal-Gal-NAc. In some embodiments, the antibody binds to the Lewis related antigens, such as but not limited to, Lewis^{Y}, Sialyl Lewis^{X}, Sialyl Lewis^{A}. In some embodiments, the antibody binds to the carbohydrate structures of glycosphingolipids classified in series, such as but not limited to, -Globo, -Isoglobo, -Ganglio, -Isoganglio, -Lacto, -Neolacto, Lactoganglio, -Muco, -Neogala, -Mollu, -Arthro, -Schisto, and -Spirometo. In some embodiments, the antibody binds to gangliosides, such as but not limited to, GD1, GD2, GD3, GM1, GM2, fucosyl Gm1, Neu5GcGm3, and polysialic acid.

In some embodiments, the compositions can also comprise one or more buffers, stabilizers, emulsifiers, or any combination thereof. In some embodiments, the compositions comprise water.

In some embodiments, the compositions comprise one or more cell penetration compounds. In some embodiments, the cell penetration compounds comprise hypotonic buffer solutions; organic compounds, such as but not limited to methanol, ethanol, acetone, toluene, DMSO, and alkyltrimethylammonium bromide; detergents, such as but not limited to saponin, TritonX-100, Tween-20, Tween-80, digitonin, sodium dodecyl sulfate (SDS); and pore forming cytolysins, such as but not limited to beta-hemoytic cytolysins such as streptolysin-O (SLO) and perfirngolysin-O (PFO); or any combination thereof. In some embodiments, the composition comprises at least one cell penetration agent selected from the group consisting of hypotonic buffer solutions, methanol, ethanol, acetone, toluene, DMSO, alkyltrimethylammonium bromide, saponin, TritonX-100, Tween-20, Tween-80, digitonin, sodium dodecyl sulfate (SDS), beta-hemoytic cytolysins, streptolysin-O (SLO), perfirngolysin-O (PFO); or any combination thereof.

Accordingly, in some embodiments, methods of inhibiting fucosyltransferase are provided. As used herein, “fucosyltransferase” refers to an enzyme that catalyzes the addition of fucose onto a target, such as a protein. Examples of fucosyltransferases include, but are not limited to, alpha 1,6 fucosyltransferase, which can also be referred to as FUT8. Thus, in some embodiments, the fucosyltransferase that is inhibited is FUT8. In some embodiments, the method comprises contacting the fucosyltransferase with a composition comprising purpurogallin, hexachlorophene, acriflavinum, baicalein, epicatechin monogallate, epicatechin-3-monogallate, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof, or any salt thereof. The fucosyltransferase can be in a cell or in a cell free system.

Various cells can comprise fucosyltransferases. Examples of cells include, but are not limited to, CHO cell, a NSO cell, a Sp2/0 cell, a HEK293 cell, or a PER.C6 cell. These cells are non-limiting examples and other cell types and strains can be used.

In some embodiments, the cell that is contacted with the compositions and compounds described herein produce an antibody. In some embodiments, the antibody is a recombinant antibody. In some embodiments, the antibody binds to one or more of the following targets: IL-8, ErbB, ErbB 1, ErbB2, ErbB3, ErbB4, HER1, HER2, HER3, HER4, TGF, TGFB1, TGFB2, TGFB3, TGFB4, VEGF, VEGFR1, Flt-1, VEGFR2, Flk-1/KDR, VEGFR3, Flt4, RANK, TNFRSFIIA, SLAMF, SLAMF1, SLAMF2, SLAMF3, SLAMF4, SLAMF5, SLAMF6, SLAMF7, SLAMF8, PDGFRα, PDGFRβ, KIR3DL3, KIR3DP1, KIR3DL4, KIR3DL2, MIC, MICA, MICB, TNF, LTalpha, LT beta, FASL, 4-IBBL, OX40L, TNF Related Apoptosis Inducing Ligand (TRAIL), DR1, DR2, DR3, Dr4, DR5, DR6, DR7, DRB, CTLA4, PD1, CD66a, CD66b, CD66c, CD66d, CD66e, CD66f, TIM1, TIM2, TIM3, LAG, LAGS, CD2, CD3, CD19, CD20, CD22, CD25, CD27, CD30, CD 33, CD38, CD39, CD52, CD70, CD73, CD94, CD134, CD137, CD252, CD340, GalNAc-O-ser/thr, Neu5Acα2-6GalNAcα-O-Ser/Thr, SSEA-, SSEA-2, SSEA-3, SSEA-4, Gal-Gal-NAc, Lewis^{Y}, Sialyl Lewis^{X}, Sialyl Lewis^{A}, -Globo, -Isoglobo, -Ganglio, -Isoganglio, -Lacto, -Neolacto, Lactoganglio, -Muco, -Neogala, -Mollu, -Arthro, -Schisto, -Spirometo, GD1, GD2, GD3, GM1, GM2, fucosyl Gm1, Neu5GcGm3, and polysialic acid.

In some embodiments, the antibody that is being produced by the cell is adalimumab, alemtuzumab, alirocumab, atezolizumab, avelumab, belimumab, benralizumab, bevacizumab, bezlotoxumab, blinatumomab, brentuximab vedotin, burosumab, canakinumab, cantuzumab mertansine, cantuzumab ravtansine, carotuximab, certolizumab pegol, cetuximab, claudiximab, daclizumab, daratumumab, denosumab, depatuxizumab mafodotin, dinutuximab, durvalumab, elotuzumab, enfortumab vedotin, enoblituzumab, gemtuzumab ozogamicin, golimumab, guselkumab, ibalizumab, ibritumomab tiuxetan, ifabotuzumab, inebilizumab, infliximab, inotuzumab ozogamicin, ipilimumab, isatuximab, margetuximab, mepolizumab, necitumumab, nimotuzumab, nivolumab, obiloxaximab, obinutuzumab, ocrelizumab, ofatumumab, olaratumab, omalizumab, palivizumab, pembrolizumab, pertuzumab, polatuzumab vedotin, racotumomab, ramucirumab, ranibizumab, raxibacumab, rituximab, rovalpituzumab tesirine, sacituzumab, sacituzumab govitecan, sarilumab, secukinumab, siltuximab, sirukumab, teprotumumab, tildrakizumab, tocilizumab, tositumomab, trastuzumab, trastuzumab duocarmazine, trastuzumab emtansine, ublituxumab, ustekinumab, vedolizumab, or a biosimilar thereof.

A biosimilar is an antibody that has the same or similar sequence but may not be identical to the antibody that was given the name described herein. A biosimilar antibody can also be an antibody that is highly similar to and has no clinically meaningful differences from an existing FDA-approved reference product, including those described herein. A biosimilar has no clinically meaningful differences if it has no meaningful (significant) differences in terms of safety, purity, and potency (safety and effectiveness) as compared to the reference product.

Without being bound to any particular theory, by inhibiting fucosyltransferase with the compositions, the compositions can, therefore, be used to reduce levels of fucosylation of proteins in a cell. The fucosylation can be reduced on a protein that is produced by the cell. In some embodiments, the protein with reduced fucosylation is an antibody. Non-limiting examples of antibodies are described herein. In some embodiments, the compositions reduce the level of fucosylation from about 1% to about 99%. In some embodiments, the compositions reduce the level of fucosylation from about 1% to about 95%. In some embodiments, the compositions reduce the level of fucosylation from about 1% to about 90%. In some embodiments, the compositions reduce the level of fucosylation from about 1% to about 85%. In some embodiments, the compositions reduce the level of fucosylation from about 1% to about 80%. In some embodiments, the compositions reduce the level of fucosylation from about 1% to about 75%. In some embodiments, the compositions reduce the level of fucosylation from about 1% to about 70%. In some embodiments, the compositions reduce the level of fucosylation from about 1% to about 65%. In some embodiments, the compositions reduce the level of fucosylation from about 1% to about 60%. In some embodiments, the compositions reduce the level of fucosylation from about 1% to about 55%. In some embodiments, the compositions reduce the level of fucosylation from about 1% to about 50%. In some embodiments, the compositions reduce the level of fucosylation from about 1% to about 45%. In some embodiments, the compositions reduce the level of fucosylation from about 1% to about 40%. In some embodiments, the compositions reduce the level of fucosylation from about 1% to about 35%. In some embodiments, the compositions reduce the level of fucosylation from about 1% to about 30%. In some embodiments, the compositions reduce the level of fucosylation from about 1% to about 25%. In some embodiments, the compositions reduce the level of fucosylation from about 1% to about 20%. In some embodiments, the compositions reduce the level of fucosylation from about 1% to about 15%. In some embodiments, the compositions reduce the level of fucosylation from about 1% to about 10%. In some embodiments, the compositions reduce the level of fucosylation from about 1% to about 5%. In some embodiments, the compositions reduce the level of fucosylation from about 5% to about 99%. In some embodiments, the compositions reduce the level of fucosylation from about 10% to about 99%. In some embodiments, the compositions reduce the level of fucosylation from about 15% to about 99%. In some embodiments, the compositions reduce the level of fucosylation from about 20% to about 99%. In some embodiments, the compositions reduce the level of fucosylation from about 25% to about 99%. In some embodiments, the compositions reduce the level of fucosylation from about 30% to about 99%. In some embodiments, the compositions reduce the level of fucosylation from about 35% to about 99%. In some embodiments, the compositions reduce the level of fucosylation from about 40% to about 99%. In some embodiments, the compositions reduce the level of fucosylation from about 45% to about 99%. In some embodiments, the compositions reduce the level of fucosylation from about 50% to about 99%. In some embodiments, the compositions reduce the level of fucosylation from about 55% to about 99%. In some embodiments, the compositions reduce the level of fucosylation from about 60% to about 99%. In some embodiments, the compositions reduce the level of fucosylation from about 65% to about 99%. In some embodiments, the compositions reduce the level of fucosylation from about 70% to about 99%. In some embodiments, the compositions reduce the level of fucosylation from about 75% to about 99%. In some embodiments, the compositions reduce the level of fucosylation from about 80% to about 99%. In some embodiments, the compositions reduce the level of fucosylation from about 85% to about 99%. In some embodiments, the compositions reduce the level of fucosylation from about 90% to about 99%. In some embodiments, the compositions reduce the level of fucosylation from about 95% to about 99%. In some embodiments, the level of fucosylation of a protein or a pool of proteins is reduced by at least 5, 10, 20, 30, 40, 50, 60, 70, 80, 90, or 95%. In some embodiments, the compositions reduce, but do not eliminate, fucosylation. A reduction of fucosylation of a protein is based upon the level of fucosylation of the protein produced from a specific cell type, wherein the cell is contacted with the compositions and compounds described herein. For example, if an antibody is being produced from a CHO cell and then the same or similar CHO cell producing the antibody is contacted with the compositions and compounds provided for herein, the level of the fucosylation on the antibody is reduced. The percent reduction is a comparison to the antibody produced from the CHO cell that is not contacted with the compositions and compounds described herein.

In some embodiments, methods of inhibiting or reducing fucosylation of a protein in a cell are provided. In some embodiments, the methods comprise contacting the cell with any of the compositions or compounds provided herein. In some embodiments, the cell is contacted with the composition in vitro or in vivo. In some embodiments, the composition inhibits fucosylation by inhibiting a fucosyltransferase.

In some embodiments, the cell is any of the cell types disclosed herein, although the cell types provided herein are non-limiting examples. In some embodiments, the cell produces an antibody. In some embodiments, the antibody is a recombinant antibody. In some embodiments, the antibody binds to one or more of the following targets: ErbB, ErbB 1, ErbB2, ErbB3, ErbB4, HER1, HER2, HER3, HER4, TGF, TGFB1, TGFB2, TGFB3, TGFB4, VEGF, VEGFR1, Flt-1, VEGFR2, Flk-1/KDR, VEGFR3, Flt4, RANK, TNFRSFIIA, SLAMF, SLAMF1, SLAMF2, SLAMF3, SLAMF4, SLAMF5, SLAMF6, SLAMF7, SLAMF8, PDGFRα, PDGFRβ, KIR3DL3, KIR3DP1, KIR3DL4, KIR3DL2, MIC, MICA, MICB, TNF, LTalpha, LT beta, FASL, 4-IBBL, OX40L, TNF Related Apoptosis Inducing Ligand (TRAIL), DR1, DR2, DR3, Dr4, DR5, DR6, DR7, DR8, CTLA4, PD1, CD66a, CD66b, CD66c, CD66d, CD66e, CD66f, TIM1, TIM2, TIM3, LAG, LAG3, CD2, CD3, CD19, CD20, CD22, CD25, CD27, CD30, CD 33, CD38, CD39, CD52, CD70, CD73, CD94, CD134, CD137, CD252, CD340, GalNAc-O-ser/thr, Neu5Acα2-6GalNAcα-O-Ser/Thr, SSEA-, SSEA-2, SSEA-3, SSEA-4, Gal-Gal-NAc, Lewis^{Y}, Sialyl Lewis^{X}, Sialyl Lewis^{A}, -Globo, -Isoglobo, -Ganglio, -Isoganglio, -Lacto, -Neolacto, Lactoganglio, -Muco, -Neogala, -Mollu, -Arthro, -Schisto, -Spirometo, GD1, GD2, GD3, GM1, GM2, fucosyl Gm1, Neu5GcGm3, and polysialic acid.

In some embodiments, the antibody is adalimumab, alemtuzumab, alirocumab, atezolizumab, avelumab, belimumab, benralizumab, bevacizumab, bezlotoxumab, blinatumomab, brentuximab vedotin, burosumab, canakinumab, cantuzumab mertansine, cantuzumab ravtansine, carotuximab, certolizumab pegol, cetuximab, claudiximab, daclizumab, daratumumab, denosumab, depatuxizumab mafodotin, dinutuximab, durvalumab, elotuzumab, enfortumab vedotin, enoblituzumab, gemtuzumab ozogamicin, golimumab, guselkumab, ibalizumab, ibritumomab tiuxetan, ifabotuzumab, inebilizumab, infliximab, inotuzumab ozogamicin, ipilimumab, isatuximab, margetuximab, mepolizumab, necitumumab, nimotuzumab, nivolumab, obiloxaximab, obinutuzumab, ocrelizumab, ofatumumab, olaratumab, omalizumab, palivizumab, pembrolizumab, pertuzumab, polatuzumab vedotin, racotumomab, ramucirumab, ranibizumab, raxibacumab, rituximab, rovalpituzumab tesirine, sacituzumab, sacituzumab govitecan, sarilumab, secukinumab, siltuximab, sirukumab, teprotumumab, tildrakizumab, tocilizumab, tositumomab, trastuzumab, trastuzumab duocarmazine, trastuzumab emtansine, ublituxumab, ustekinumab, vedolizumab, or a biosimilar thereof. The level of fucosylation inhibition or reduction can be as described herein.

In some embodiments, methods of inhibiting fucosyltransferase are provided. In some embodiments, the methods comprise contacting the fucosyltransferase with any of the compositions or compounds provided herein. In some embodiments, the fucosyltransferase is in a cell. In some embodiments, the cell is any of the cell types provided for herein. In some embodiments, the cell produces an antibody. In some embodiments, the antibody is a recombinant antibody. In some embodiments, the antibody is any of the antibodies disclosed herein. In some embodiments, the antibody binds to any of the targets or antigens provided for herein.

In some embodiments, methods of producing an antibody are provided. In some embodiments, methods of producing an antibody with reduced fucosylation are provided. In some embodiments, the methods comprise contacting a cell producing an antibody with any of the compositions and compounds provided herein. The cell can be contacted with the compositions and compounds in vitro or in vivo. The cell producing the antibody is not limited to any particular cell type and can, for example, be any of the cell types provided for herein. In some embodiments, the antibody is a recombinant antibody. In some embodiments, the antibody is any of the antibodies provided for herein. In some embodiments, the antibody is directed towards any of the provided for disclosed herein.

In some embodiments, any of the methods provided herein comprise purifying the antibody with reduced fucosylation. Antibodies can be purified by, for example, column chromatography, precipitation, and the like. Other methods of purification include, but are not limited to, size exclusion chromatography, ammonium sulfate precipitation, ion exchange chromatography, immobilized metal chelate chromatography, thiophilic adsorption, melon Gel chromatography, protein A, G and L antibody-binding ligands, antibody purification with Protein A, G and L, and the like. These purification methods are non-limiting and other methods can also be used.

In some embodiments, methods of producing an antibody with an altered glycosylation pattern are provided. In some embodiments, the methods comprise contacting a cell producing the antibody with any of the compositions or compounds provided herein. In some embodiments, the composition contacts the cell in vitro or in vivo. In some embodiments, the cell is any of the cell types provided herein. In some embodiments, the antibody is a recombinant antibody. In some embodiments, the antibody is any of the antibodies disclosed herein. In some embodiments, the antibody is directed towards any of the antigens disclosed herein.

The antibodies produced herein can also be used to in methods of treating subjects for the various conditions for which they were developed, such as cancer, auto-immune diseases, and the like. Thus, in some embodiments, methods of treating a subject are provided. In some embodiments, the methods comprise contacting a cell producing a therapeutic antibody with a compound or composition as described herein to produce a therapeutic antibody with reduced fucosylation. Non-limiting examples of therapeutic antibodies are described herein. The antibody with reduced fucosylation can be isolated and be prepared in a pharmaceutical composition or formulation suitable for administration to a subject in need thereof. In some embodiments, the subject is treated for cancer or an auto-immune disease, such as rheumatoid arthritis, Crohn's disease, ulcerative colitis, ankylosing spondylitis, Behçet's disease, fistulizing diease, hidradenitis suppurativa, juvenile idiopathic arthritis, psoriasis, psoriatic arthritis, and relapsing polychondritis.

The embodiments are now described with reference to the following examples. These examples are provided for the purpose of illustration only and the embodiments should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLES

The following examples are illustrative, but not limiting, of the compositions and methods described herein. Other suitable modifications and adaptations known to those skilled in the art are within the scope of the following embodiments.

### Example 1: Inhibition of Fucosylation by Selected Compounds

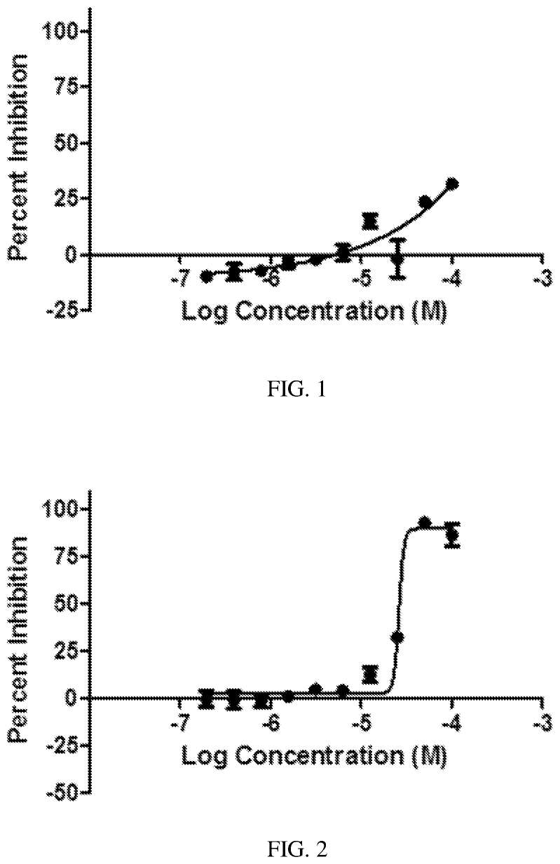

The compounds illustrated in this example were found to inhibit FUT8 activity, which demonstrated that the compounds can inhibit a fucosyltransferase. The compounds illustrated in below were found to be effective to inhibit FUT8 activity and would be expected to reduce fucosylation of antibodies being produced from a cell containing a fucosyltransferase, such as FUT8. Briefly, a compound was combined with fucosyltransferase (FUT-8) and appropriate substrates and incubated for 1 hour. The reaction was terminated, and a detection reagent mixture was added to detect the amount of fucosylation of the substrate protein. The inhibition values for each compound are shown in Table 2 and also depicted in FIGS. 1-19.

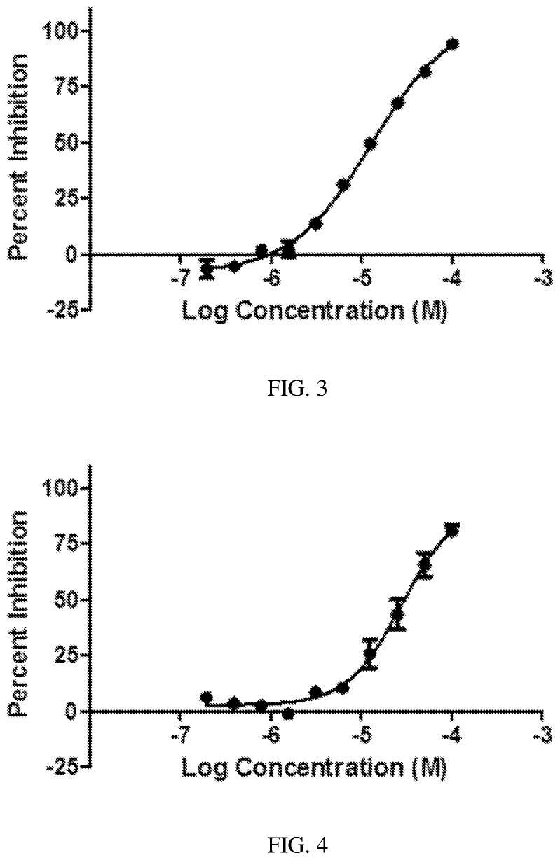

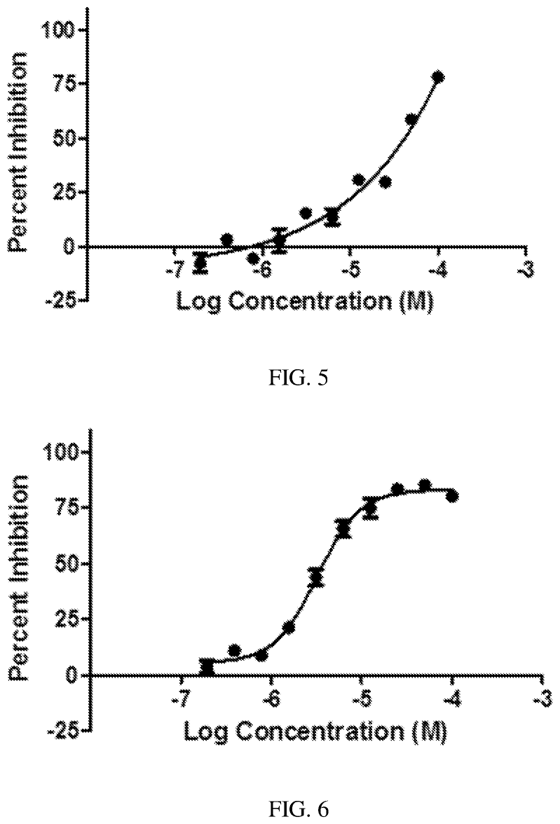

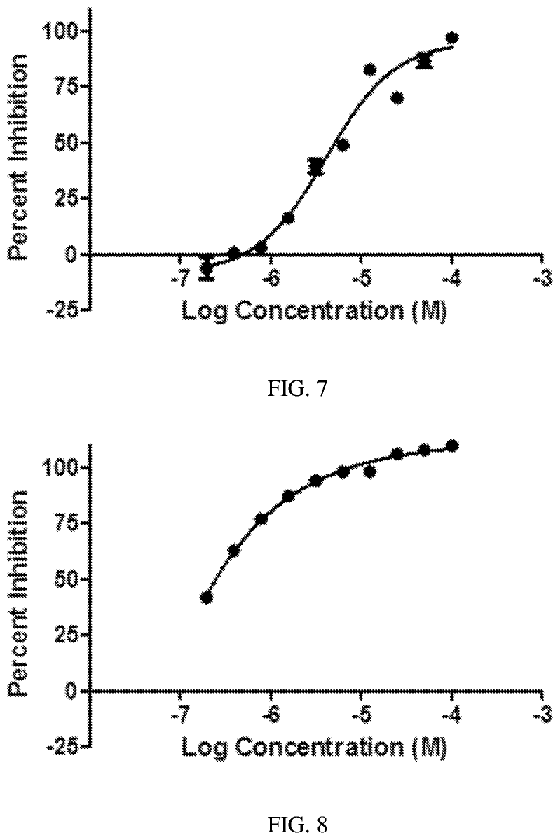

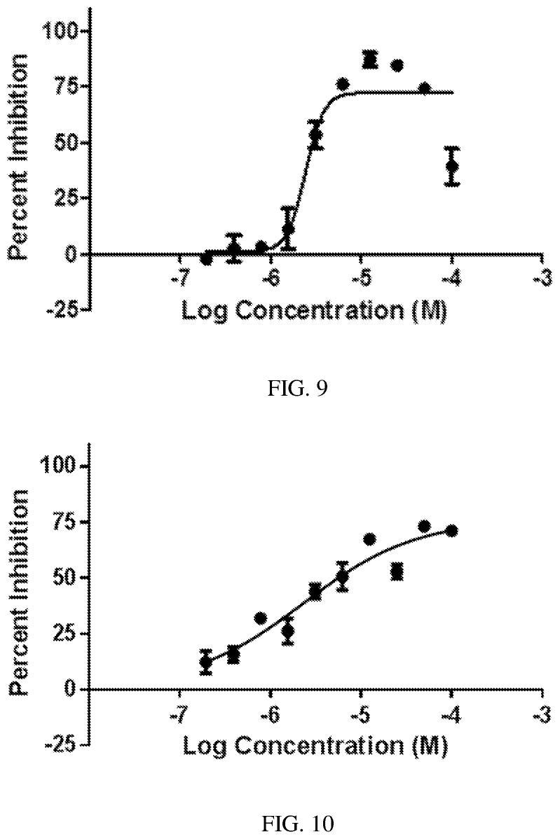

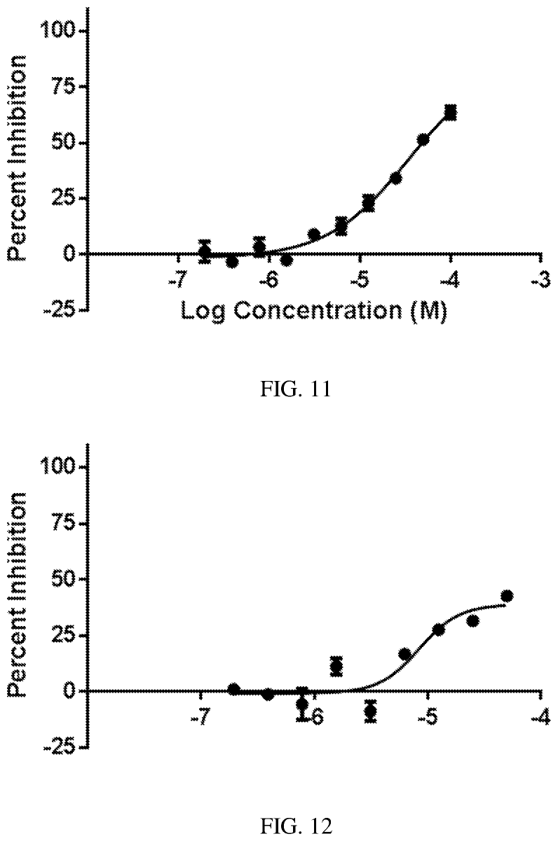

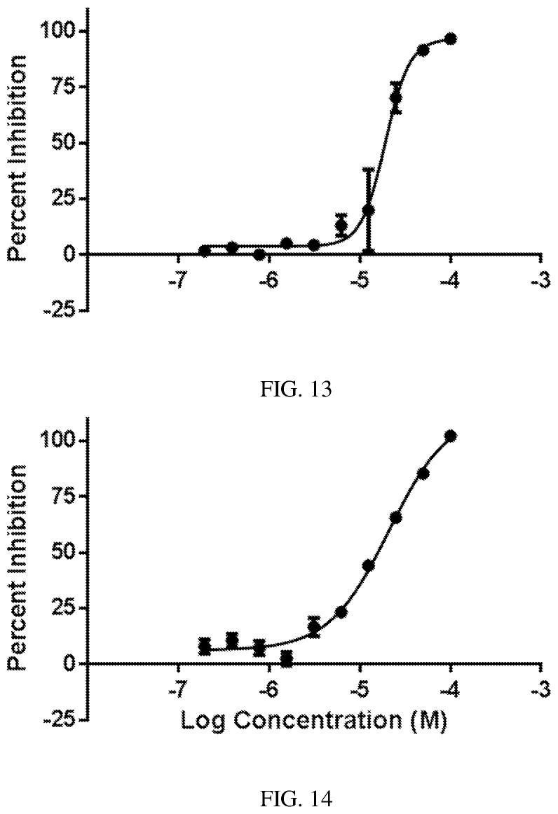

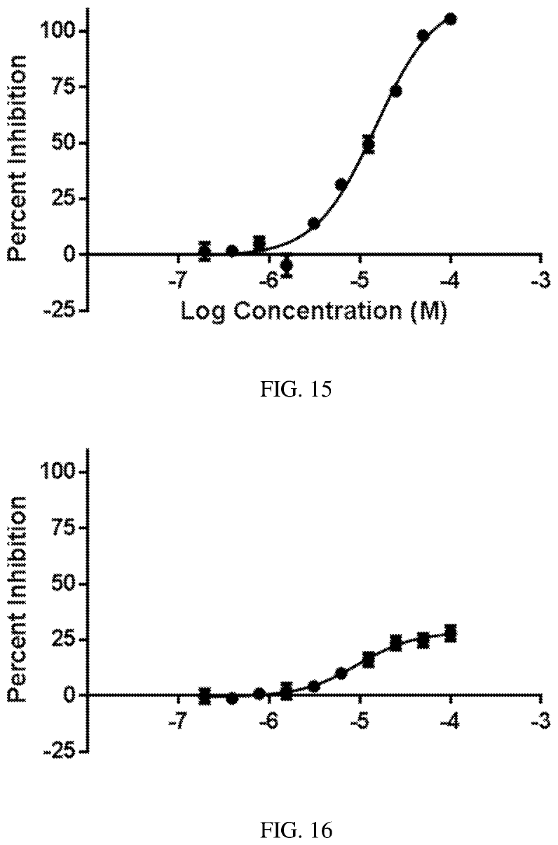

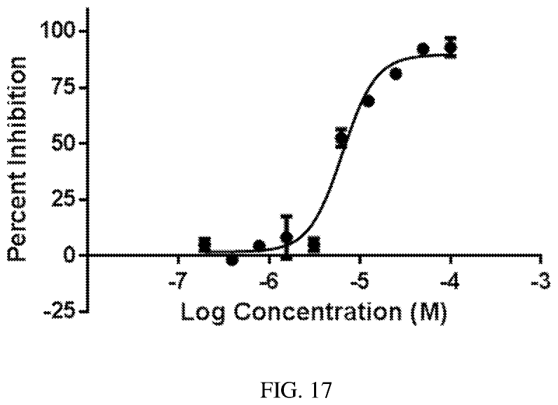

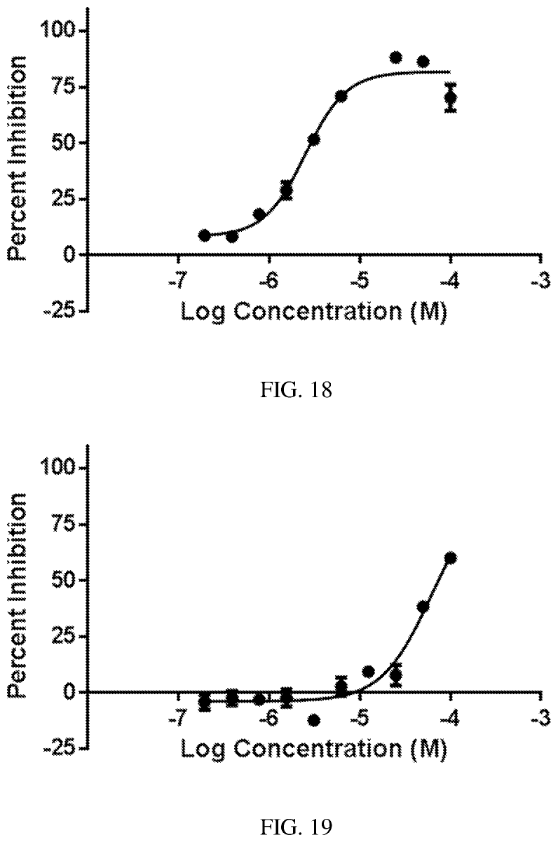

TABLE 2
Inhibition Values

| | Primary Screen | IC50 Value | |
|---|---|---|---|
| Compound Name | (10 μM) | (μM) | Graph |
| Purpurogallin | 30% | >10 | FIG. 1 |
| Hexachlorophene | 40% | >10 | FIG. 2 |
| Acriflavinium | 47% | >10 | FIG. 3 |
| Baicalein | 29% | — | |
| Epicatechin Monogallate | 25% | >10 | FIG. 4 |
| Epicatechin-3-Monogallate | 39% | >10 | FIG. 5 |
| Epicatechin-3,5-Digallate | 83% | <10 | FIG. 6 |
| Theaflavin Monogallate | 88% | <10 | FIG. 7 |
| Tannic Acid | 93% | <10 | FIG. 8 |
| Methacycline | 82% | <10 | FIG. 9 |
| Anthralin | 28% | >10 | |
| Mitoxanthrone Hydrocloride | 49% | >10 | FIG. 10 |
| Hycanthone | 27% | >10 | FIG. 11 |
| Ethcridine Lactate | 26% | >10 | FIG. 12 |
| Aurin Tricarboxylic Acid | 34% | >10 | FIG. 13 |
| Carboplatin | 45% | >10 | FIG. 14 |
| Cisplatin | 60% | >10 | FIG. 15 |
| Primuletin | 30% | >10 | |
| Chrysin | 30% | >10 | |
| Diometin | 35% | >10 | FIG. 16 |
| Suramin | 70% | <10 | FIG. 17 |
| Hematin | 82% | <10 | FIG. 18 |
| Gossypol | 26% | >10 | FIG. 19 |

### Example 2: Fucosyltransferase Inhibitors Reduce Antibody Fucosylation

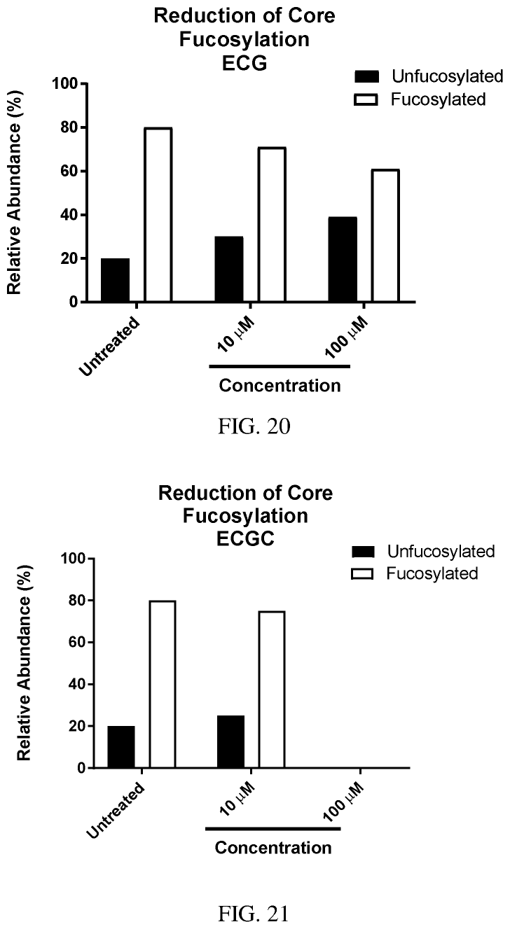

The compounds illustrated in this example were found to reduce the level of fucosylation of antibodies being produced by a cell containing a fucosyltransferase, such as FUT8. Briefly, Epicatechin Monogallate (ECG), Epicatechin-3-Monogallate (ECGC) and Aurin Tricarboxylic Acid (ATA) were included in the cell culture media and added to CHO cells producing an anti-IL8 IgG monoclonal antibody (mAb) obtained from ATCC (ATCC CRL-12445), which is also described in U.S. Pat. No. 6,025,158, which is hereby incorporated by reference in its entirety. After a specified period of time the cells were harvested and the anti-IL8 mAb purified by standard methods. Purified anit-IL8 mAbs were then reduced by the addition of dithiothreitol and subjected to LC-MS analysis to determine the level of fucosylation. The decrease in the level of anti-IL8 fucosylation are depicted in FIG. 20 AND FIG. 21. These results demonstrate that inhibiting fucosyltransferase leads to a reduction of antibody fucosylation. Furthermore, ECG demonstrated a dose dependent decrease in the core fucosylated species with a concomitant rise in the afucosylated species. At the maximum concentration of ECG (100 μM) a 25% reduction in fucosylation was noted whereas at 10-fold lower concentration a 13% reduction was achieved. ECGC at 10 μM achieved a 6% reduction in fucosylation. For this antibody, ATA did not alter the fucosylation level at either concentration tested (data not shown). These results demonstrate that using different types of fucosyltransferase inhibitors can lead to a reduction in antibody fucosylation.

This description is not limited to the particular processes, compositions, or methodologies described, as these may vary. The terminology used in the description is for the purpose of describing the particular versions or embodiments only, and it is not intended to limit the scope of the embodiments described herein. Unless defined otherwise, all technical and scientific terms used herein have the same meanings as commonly understood by one of ordinary skill in the art. In some cases, terms with commonly understood meanings are defined herein for clarity and/or for ready reference, and the inclusion of such definitions herein should not necessarily be construed to represent a substantial difference over what is generally understood in the art. However, in case of conflict, the patent specification, including definitions, will prevail.

From the foregoing, it will be appreciated that various embodiments of the present disclosure have been described herein for purposes of illustration, and that various modification can be made without departing from the scope and spirit of the present disclosure. Accordingly, the various embodiments disclosed herein are not intended to be limiting.

What is claimed is:

1. A method of inhibiting fucosyltransferase comprising contacting the fucosyltransferase with a compound of, or composition comprising, epicatechin monogallate (ECG), epicatechin-3-monogallate (ECGC), purpurogallin, hexachlorophene, acriflavinum, baicalein, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof.

2. The method of claim 1, wherein the fucosyltransferase is in a cell.

3. The method of claim 1, wherein the cell produces an antibody.

4. The method of claim 3, wherein the antibody is a recombinant antibody.

5. The method of claim 3, wherein the antibody binds to one or more of the following targets: IL8, ErbB, ErbB 1, ErbB2, ErbB3, ErbB4, HER1, HER2, HER3, HER4, TGF, TGFB1, TGFB2, TGFB3, TGFB4, VEGF, VEGFR1, Flt-1, VEGFR2, Flk-1/KDR, VEGFR3, Flt4, RANK, TNFRSFIIA, SLAMF, SLAMF1, SLAMF2, SLAMF3, SLAMF4, SLAMF5, SLAMF6, SLAMF7, SLAMF8, PDGFRα, PDGFRβ, KIR3DL3, KIR3DP1, KIR3DL4, KIR3DL2, MIC, MICA, MICB, TNF, LTalpha, LT beta, FASL, 4-IBBL, OX40L, TNF Related Apoptosis Inducing Ligand (TRAIL), DR1, DR2, DR3, Dr4, DR5, DR6, DR7, DRB, CTLA4, PD1, CD66a, CD66b, CD66c, CD66d, CD66e, CD66f, TIM1, TIM2, TIM3, LAG, LAGS, CD2, CD3, CD19, CD20, CD22, CD25, CD27, CD30, CD 33, CD38, CD39, CD52, CD70, CD73, CD94, CD134, CD137, CD252, CD340, GalNAc-O-ser/thr, Neu5Acα2-6GalNAcα-O-Ser/Thr, SSEA-, SSEA-2, SSEA-3, SSEA-4, Gal-Gal-NAc, Lewis^{Y}, Sialyl Lewis^{X}, Sialyl Lewis^{A}, -Globo, -Isoglobo, -Ganglio, -Isoganglio, -Lacto, -Neolacto, Lactoganglio, -Muco, -Neogala, -Mollu, -Arthro, -Schisto, -Spirometo, GD1, GD2, GD3, GM1, GM2, fucosyl Gm1, Neu5GcGm3, and polysialic acid.

6. The method of claim 3, wherein the antibody that is being produced by the cell is adalimumab, alemtuzumab, alirocumab, atezolizumab, avelumab, belimumab, benralizumab, bevacizumab, bezlotoxumab, blinatumomab, brentuximab vedotin, burosumab, canakinumab, cantuzumab mertansine, cantuzumab ravtansine, carotuximab, certolizumab pegol, cetuximab, claudiximab, daclizumab, daratumumab, denosumab, depatuxizumab mafodotin, dinutuximab, durvalumab, elotuzumab, enfortumab vedotin, enoblituzumab, gemtuzumab ozogamicin, golimumab, guselkumab, ibalizumab, ibritumomab tiuxetan, ifabotuzumab, inebilizumab, infliximab, inotuzumab ozogamicin, ipilimumab, isatuximab, margetuximab, mepolizumab, necitumumab, nimotuzumab, nivolumab, obiloxaximab, obinutuzumab, ocrelizumab, ofatumumab, olaratumab, omalizumab, palivizumab, pembrolizumab, pertuzumab, polatuzumab vedotin, racotumomab, ramucirumab, ranibizumab, raxibacumab, rituximab, rovalpituzumab tesirine, sacituzumab, sacituzumab govitecan, sarilumab, secukinumab, siltuximab, sirukumab, teprotumumab, tildrakizumab, tocilizumab, to situmomab, trastuzumab, trastuzumab duocarmazine, trastuzumab emtansine, ublituxumab, ustekinumab, vedolizumab, or a biosimilar thereof.

7-12. (canceled)

13. A method of producing an antibody with reduced fucosylation, the method comprising contacting a cell producing the antibody with a compound of, or a composition comprising, epicatechin monogallate, epicatechin-3-monogallate, purpurogallin, hexachlorophene, acriflavinum, baicalein, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof.

14. The method of claim 13, wherein the cell is contacted with the compound or composition in vitro or in vivo.

15. The method of claim 13, wherein the compound or composition inhibits fucosyltransferase.

16. The method of claim 13, wherein the antibody is secreted from the cell.

17. The method of claim 16, wherein the compound or composition contacts the fucosyltransferase prior to the secretion of the antibody.

18. The method of claim 13, wherein the produced antibody has a reduced level of fucosylation as compared to an antibody produced from a cell that was not contacted with the compound or composition.

19. The method of claim 13, wherein the compound or composition comprises at least one cell penetration agent selected from the group consisting of hypotonic buffer solutions, methanol, ethanol, acetone, toluene, DMSO, alkyltrimethylammonium bromide, saponin, TritonX-100, Tween-20, Tween-80, digitonin, sodium dodecyl sulfate (SDS), beta-hemoytic cytolysins, streptolysin-O (SLO), perfirngolysin-O (PFO); or any combination thereof.

20. The method of claim 13, wherein the cell is a CHO cell, a NSO cell, a Sp2/0 cell, a HEK293 cell, or a PER.C6 cell.

21-28. (canceled)

29. A composition comprising:
epicatechin monogallate, epicatechin-3-monogallate, purpurogallin, hexachlorophene, acriflavinum, baicalein, epicatechin-3,5-digallate, theaflavin monogallate, tannic acid, methacycline, anthralin, mitoxanthrone hydrochloride, hycanthone, ethcridine lactate, aurin tricarboxylic acid, carboplatin, cisplatin, primuletin, chrysin, diometin, suramin, hematin, gossypol, or any combination thereof;
a cell; and
a recombinant antibody.

30. (canceled)

31. The composition of claim 29, wherein the cell is a CHO cell, a NSO cell, a Sp2/0 cell, a HEK293 cell, or a PER.C6 cell.

32. The composition of claim 29, further comprising cell culture media.

33. The composition of claim 29, further comprising one or more antibiotics.

34. The composition of claim 29, further comprising one or more selection agents.

35. The composition of claim 29, further comprising a recombinant antibody produced and secreted from the cell.

36-40. (canceled)
